(19) United States
(12) Patent Application Publication
(10) Pub. No.: US 2025/0149131 A1
(43) Pub. Date: May 8, 2025

(54) SYSTEM AND METHOD FOR MANAGING MEDICAL QUALITY INDICATOR

(71) Applicant: Taipei Veterans General Hospital, Taipei City (TW)

(72) Inventors: Yuan-Hwa CHOU, Taipei City (TW); Yu-Cheng Luo, Taipei City (TW); Ying-Chun Jheng, Taipei City (TW)

(73) Assignee: Taipei Veterans General Hospital, Taipei City (TW)

(21) Appl. No.: 18/584,818

(22) Filed: Feb. 22, 2024

(30) Foreign Application Priority Data
Nov. 8, 2023 (WO) PCT/CN2023/130473

(51) Int. Cl. G16H 10/60

(52) U.S. Cl. CPC G16H 10/60

(57) ABSTRACT

Disclosed is a system for managing medical quality indicator, which includes a target medical information database, a data retrieving module, a data analyzing module and a data visualization module. The data retrieving module and the data analyzing module is connected to the target medical information database. The data analyzing module performs data confirmation and data exclusion to the plurality of structured data according to an operational definition, and performs computation to the plurality of structured data according to a viewing request, to obtain a set of statistical data. The data visualization module is connected to the data analyzing module and is used to display the set of statistical data through a user interface.

### FIELD OF THE INVENTION

The present invention relates to a system and a method for managing a medical quality indicator.

### BACKGROUND OF THE INVENTION

In hospitals, medical quality indicators are usually calculated with program assistance, and the work includes: obtaining data from the subsystem database, retrieving data at high frequencies and viewing cases in different forms.

However, data from different sources are scattered in different subsystem databases. High-frequency retrieval of data can easily lead to system instability, but reducing the frequency of retrieval of data will also result in the difficulty of accession to real-time information.

Therefore, there is still a need for a more effective method and system for managing and retrieving data.

### SUMMARY OF THE INVENTION

The present invention provides a system for managing a medical quality indicator, which comprises: a target database for medical data; a data retrieving module connected to the target database, wherein in response to a request for viewing a medical quality indicator or based on an update frequency of the medical quality indicator set in a data update frequency control table, the data retrieving module obtains required data from one or more primary database for medical data according to an operational definition of the medical quality indicator, and preprocesses the required data and stores it as a plurality pieces of structured data of the medical quality indicator in the target database, each piece of the structured data correspondingly storing a patient data of a patient, a clinical index data of the patient and an indicator factor data of the medical quality indicator; and the data retrieving module, based on a hot data interval of the medical quality indicator set in the data update frequency control table, determines whether only a piece of data of the required data present in the hot data interval is obtained, or obtains a piece of data of the required data present in the hot data interval according to the update frequency; a data analyzing module connected to the target database, which performs a data confirmation and data exclusion with respect to the plurality pieces of structured data according to the operational definition, and performs a computation to the plurality pieces of structured data according to the viewing request to obtain a set of statistical data; and a data visualization module connected to the data analyzing module and used for displaying the set of statistical data through a user interface.

In one embodiment of the present invention, the operational definition defines one or more data items required for evaluating the medical quality indicator, and the data retrieving module obtains the required data from one or more corresponding primary databases according to the data items.

In one embodiment of the present invention, the patient data includes medical record number, hospitalization number, inspection sheet number, name, gender, birthday, or a combination thereof.

In one embodiment of the present invention, the clinical index data includes outpatient, emergency department, inpatient, or a combination thereof.

In one embodiment of the present invention, a cold data interval is further set in the data update frequency control table, and the data retrieving module first determines whether both a piece of data of the required data present in the hot data interval and a piece of data of the required data present in the cold data interval are stored as structured data in the target database, if negative, then obtains data not stored stores it as structured data in the target database, and if affirmative, then obtains only the piece of data of the required data present in the hot data interval, and stores it as structured data in the target database, or accordingly updates the plurality of structured data stored in the target database.

In one embodiment of the present invention, the system further comprises a mission scheduling module connected to the data retrieving module and used for arranging the data retrieving module's data retrieval or update with respect to a plurality of medical quality indicators, the mission scheduling module having a mission description list and a schedule control unit.

In one embodiment of the present invention, the mission description list records an update frequency, a retry frequency, a hot data interval and an upstream mission name for each of the plurality of medical quality indicators.

In one embodiment of the present invention, the schedule control unit activates the data retrieving module to perform data retrieval or update according to the mission description list.

The present invention also provides a method for managing a medical quality indicator, which comprises: providing a target medical information database, a the data retrieving module, a the data analyzing module and a the data visualization module, wherein the data retrieving module is connected to the target medical information database, the data analyzing module is connected to the target medical information database, the data visualization module is connected to the data analyzing module; using the data retrieving module to obtain required data from one or more original medical information database corresponding to a viewing request of a medical quality indicator, or according to a the update frequency of the medical quality indicator configured in a data update frequency control table, according to an operational definition of the medical quality indicator, after a data processing, storing the required data as the plurality of structured data of the medical quality indicator in the target medical information database, wherein each of the structured data correspondingly stores a patient data of a patient, a clinic index data of the patient and an indicator factor data of the medical quality indicator; using the data retrieving module to determine whether only a data located in the hot data interval in the in the required data is obtained according to a hot data interval of the medical quality indicator configured in the data update frequency control table, or a data located in the hot data interval in the required data is obtained according to the update frequency; using the data analyzing module to perform data confirmation and data exclusion to the plurality of structured data according to the operational definition, and perform a computation to the plurality of structured data according to the viewing request to obtain a set of statistical data; and using the data visualization module to display the set of statistical data through a user interface.

Other objects and advantages of the present invention are partly recorded in the following description, or can be understood through the embodiments of the present invention. It should be understood that the foregoing content of the invention and the following embodiments are only exemplary and illustrative. Description, rather than limitation of the invention as in the scope of the patent application.

### BRIEF DESCRIPTION OF THE DRAWINGS

The foregoing summary, as well as the following detailed description of the invention, will be better understood when read in conjunction with the appended drawing. In the drawings:

FIG. 1 provides a block diagram of a system according to one embodiment of the present invention.

### DESCRIPTION OF THE INVENTION

The following embodiments when read with the accompanying drawings are made to clearly exhibit the above-mentioned and other technical contents, features and effects of the present invention. Through the exposition by means of the specific embodiments, people would further understand the technical means and effects the present invention adopts to achieve the above-indicated objectives. Moreover, as the contents disclosed herein should be readily understood and can be implemented by a person skilled in the art, all equivalent changes or modifications which do not depart from the concept of the present invention should be encompassed by the appended claims.

As used in the specification and the appended claims, the singular forms “a,” “an” and “the” include plural referents unless the context clearly dictates otherwise. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

Furthermore, the ordinals recited in the specification and the claims such as “first”, “second” and so on are intended only to describe the elements claimed and imply or represent neither that the claimed elements have any proceeding ordinals, nor that sequence between one claimed element and another claimed element or between steps of a manufacturing method. The use of these ordinals is merely to differentiate one claimed element having a certain designation from another claimed element having the same designation.

Furthermore, the terms recited herein such as “on”, “above”, “over” or the like are intended not only direct contact with the other component, e.g. a substrate, but also intended indirect contact with the other component, e.g. a substrate.

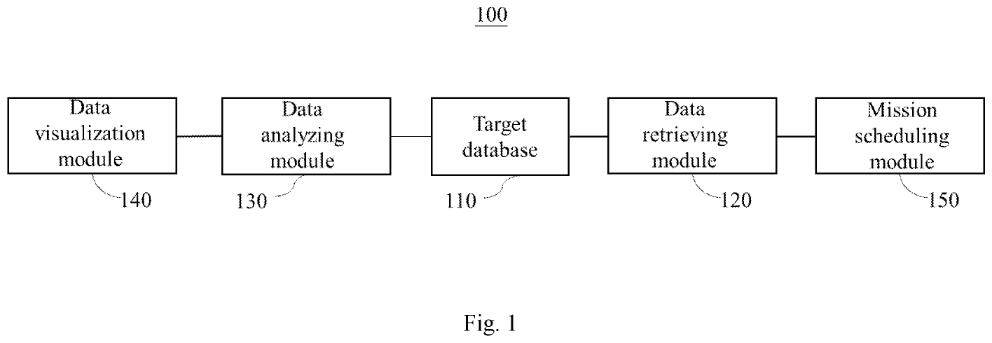

As shown in FIG. 1, the present invention provides a system 100 for managing a medical quality indicator, which includes a target database 110, a data retrieving module 120, a data analyzing module 130, a data visualization module 140, and a mission scheduling module 150. The target database 110 stores medical data. The data retrieving module 120 is connected to the target database 110. In response to a request for viewing a medical quality indicator or based on an update frequency of the medical quality indicator set in a data update frequency control table, the data retrieving module 120 obtains required data from one or more primary database for medical data according to an operational definition of the medical quality indicator, and preprocesses the required data and stores it as a plurality pieces of structured data of the medical quality indicator in the target database 110. Each piece of the structured data correspondingly storing a patient data of a patient, a clinical index data of the patient and an indicator factor data of the medical quality indicator. The data retrieving module 120, based on a hot data interval of the medical quality indicator set in the data update frequency control table, determines whether only a piece of data of the required data present in the hot data interval is obtained, or obtains a piece of data of the required data present in the hot data interval according to the update frequency.

The data analyzing module 130 connected to the target database 110, which performs a data confirmation and data exclusion with respect to the plurality pieces of structured data according to the operational definition, and performs a computation to the plurality pieces of structured data according to the viewing request to obtain a set of statistical data.

The data visualization module 140 is connected to the data analyzing module 130 and used for displaying the set of statistical data through a user interface.

Each module or database mentioned above may independently include a storage device, a processor and/or a communication chip. Storage devices include hard drives, flash drives, memories, memory cards, etc. Processors including integrated circuits such as microcontroller units, microprocessors, digital signal processors, application special integrated circuits (ASICs), logic circuits or other similar components or a combination of the above components. Communication chips can be implemented as long-term evolution systems (LTE), Worldwide Interoperability for Microwave Access System (WiMAX), Wireless Fidelity System (Wi-Fi), Bluetooth transmission, Global Mobile Communications (GSM) or Personal Handheld Phone System (PHS), etc.

In order to solve the problems in the prior art, the present invention proposes a medical quality indicator management system that can manage a large amount of real-time report data on a single platform for various management situations in the hospital. The present invention through “cold and hot data interval” and “upstream and downstream” data sharing, and optimize the “work schedule index mode” to simplify the query process and reduce management costs, so as to manage a large number of real-time reports on a single platform in various management scenarios in the hospital data. Hot data allows users to perform updates only for important hot data (such as data within 1 week or 1 month) every time they view a report. Cold data does not perform data update every time they view a report. This part of data may be (data from half a year ago or data from a year ago). When the downstream data needs to perform data retrieval, there is no need to re-create the upstream data (already existing), only the existing upstream data report needs to be passed through, obtain the required data and performs detailed data extraction. There is no need to repeatedly run the host database resources, and the resources and security of the host database can be ensured.

Taking the surgery list as an example, the report presents the data of the past three years. If all data is queried in each report, the search time for all data is about 15 minutes. And for the analysis of surgical pathology orders, the data of 3 years takes about 3-4 hours.

Taking surgical pathology order analysis as an example, through the upstream and downstream data structures, performs sub-specialty surgery list and surgical pathology order analysis, each search report does not need to perform the whole hospital surgery list, that is to say, each user uses Reporting once, it can save about 15 minutes to re-establish the hospital's surgery list report data, and the host can reduce the search resources of about 330,000 items.

Taking the analysis of surgical pathology orders as an example, most of the data in the database are established immediately after the operation. Even if there is a backup or modification of the data, most of them will not change after 7 days after the record is generated. According to this logic, the hot data interval of the report is set to 7 days, which means that when the data is updated, only seven days of data will be updated. For example, if data is updated on 2023 Oct. 10, only data during seven days 2023 Oct. 3 to 2023-10 will be updated.

Taking the response rate of patient examination critical value as an example, doctors are required to use official mobile phones, hospital websites (SMSOT), or medical operations within 24 hours after receiving a text message notifying patients of abnormal test results or critical value. The system reports query responses. That is to say, as long as more than 24 hours have passed since the SMS notification was sent, regardless of whether the doctor has responded, the records will be regarded as unanswered. At the same time, in hospital practice, the monitoring will be open for 7 days, there is no reply within 24 hours but there is a record of reply within 7 days. From the above definition, the operating characteristics of the indicator can be set: cold data is the system record of all check exception records seven days ago (data that has exceeded 7 days does not need to be checked every times updated), hot data is the data record within 7 days (must confirm whether there is a reply), the update frequency of hot data (once every 8 hours). For example, if data is updated on 2023 Oct. 10, only hot data from 2023 Oct. 3 to 2023 Oct. 10 is updated, and the cold data before 2023 Oct. 3 do not need to be updated.

Taking the operation list as an example, it can be divided into three reports: the whole hospital operation list, the sub-specialty operation list, and the surgical pathology order analysis. The whole hospital operation list is focused on the top managers of the hospital to understand the operation status of the whole hospital without going into detailed level of each department. The operation list of each department is for managers of each department to understand the status of the entire department. It is only for the department, and each department has its own independent report. The analysis of surgical pathology orders is for the quality management unit or each department. Managers of specific operations need to go through the report to understand the progress of each of the operation records in detail when encountering patient safety concerns or medical quality incidents. The hospital operation list is the highest level and covers all operation records in the hospital, but this part does not require detailed data for each operation (surgical method, reason, anesthesia method, etc.). The sub-specialty operation list and surgical pathology order analysis are both performed according to the list of the whole hospital operation list to perform detailed analysis. The hospital-wide surgery list report is the upstream data of the other two reports. The sub-specialty surgery list and surgical pathology order analysis are downstream data. When the downstream data needs to perform data extraction, there is no need to re-create the upstream data (already existing). It only needs to go through the existing upstream data report and obtain the required data performs detailed data retrieval. There is no need to repeatedly run the host database resources, and the resources and security of the host database can be guaranteed.

Taking the surgery list as an example, the report presents the data of the past three years. If all data is queried in each report, the search time for all data is about 15 minutes. For the analysis of surgical pathology orders, the data for 3 years takes about 3-4 hours.

Taking surgical pathology order analysis as an example, through the upstream and downstream data structures, performs sub-specialty surgery list and surgical pathology order analysis, each search report does not need to perform the hospital-wide surgery list, that is, each time when an user uses the report, it can save about 15 minutes to re-establish the hospital's surgery list report data, and the host can reduce about 330,000 search resources.

Taking the analysis of surgical pathology orders as an example, most of the data in the database are created immediately after the operation. Even if there is a waiting list or data modification, most of them will not change within 7 days of the record being generated. According to this logic, the hot data interval of the report is set to 7 days, which means that each time the data is updated, only seven days of data will be updated.

The mechanism of cold hot data allows only 7 days of data to be updated each time the report data is updated, which can be completed in about 1-2 minutes. The original 3-4 hours be shortened to 1-2 minutes, and real-time reports can be produced within a short period of time. Meanwhile, it can reduce the host database search resources by millions of times (millions of data searches are reduced to 2,000 data searches). The original report production method is limited by host resources and agility. Due to limitations such as data security, indicator reports cannot present data immediately, which is very limited in applications in the medical field. Through cold hot data and upstream and downstream data mechanisms of the present invention, the report data collection time that originally required several hours can be shortened (approximately millions of search resources), shortened to 1-2 minutes (about 2,000 search resources). This can quickly produce real-time reports within a period of time to meet the needs of actual clinical fields.

In an embodiment of the present invention, the quality control center refers to the scalable medical quality indicator management system and its operation method proposed by the present invention, by modularizing each system and empowering the entire medical quality indicator management system. It is scalable. According to the indicator operational definition, it automatically retrieves the digitized data in the database and performs computation, and then automatically transmits the indicator results to the visual management platform (SASVA) for users to view in real time.

Therefore, the intelligent indicator function includes automatic acquisition, computation, and presentation of indicator information; real-time presentation of data (the update frequency of the indicator, updated every 8 hours); graphical user interface; through click and chart method to present data.

In an embodiment of the present invention, the present invention quickly establishes a fully automated COVID dedicated ward battle situation board and case tracking mechanism, successfully achieving standardized information, automated data processing, visual presentation and other important items, and in a short time Quickly go online within 10 seconds, and performs adjustments at any time based on clinical needs.

As shown in FIG. 1, the medical quality indicator management system 100 of the present invention includes target medical information database 110, the data retrieving module 120, the data analyzing module 130, the data visualization module 140 and the mission scheduling module 150. The data retrieving module 120 is connected to the target medical information database 110, the data analyzing module 130 is connected to the target medical information database 110, the data visualization module 140 is connected to the data analyzing module 130, the mission scheduling module 150 is connected to the data retrieving module 120. The data retrieving module 120 includes scalability database extract fetching interface for different medical index database reading interface to retrieve data, the reading interface at least includes SQL syntax and API communication interface. At the same time, define data type, data content calculation, field naming comparison and data type Integration, etc. are converted into a complete data. The data retrieving module 120 converts the complete data after the data retrieving module to create a data set of “patient-clinic index-indicator factor”, and at the same time matches the subsequent analysis module requirements to provide scalable definitions, for each data defined indicator, additional descriptive information other than factor is provided, output and written to the destination database.

The data retrieving module 120 includes the following control items: data access method control table, data update frequency control table, data computation logic control table, data output format comparison table. the data retrieving module 120 through data access method control table, can be interfaced Different data sources include relational databases, such as Oracle, SQL server, DB2, etc. that support ETL, extract, transform and load operations through SQL syntax that performs data; or is a file type data sources, such as Excel files, TXT files, XML files and other data sources. The data retrieving module can pass through multiple control items, interface with multiple data sources and multiple terminal settings. The data source can be accorded to usage requirements defined as cold hot data, through data update frequency control table defines the interval between data update frequency and cold hot data. The data retrieving module 120 determines whether the data is new data each time it updates data. If it is new data, regardless of cold hot data, it is controlled by through data computation logic. The table and data output format comparison table are output to the destination database storage. If it is old data and is cold data, data processing is not performed to improve the performance of data processing. If it is old data and is hot data, then through data computation logic control table after table processing with the data output format, output to the destination database for storage.

The data retrieving module 120, according to data outputs the data type provided by the format comparison table, names and integrates custom multiple fields performs data, and outputs data in a standardized format and stores it in the destination database.

The data retrieving module 120 corresponds to a the viewing request of a medical quality indicator, or according to a the update frequency of the medical quality indicator configured in a data update frequency control table, according to an operational definition of the medical quality indicator to one or more original medical information database 110 obtain required data, the operational definition defines a data item that evaluates the medical quality indicator required, and the data retrieving module according to the data item obtains the required data from corresponding original medical information database, through a data processing After that, the plurality of structured data of the medical quality indicator stored as the target medical information database 110, each of the structured data correspondingly stores a patient's patient data (medical record number, hospitalization number, test order number, name, gender, birthday, diagnosis code, medication record, treatment record, examination record, outpatient emergency hospitalization record or combination thereof), a clinic index data of the patient (clinic, emergency, hospitalization or combination thereof) and the medical quality indicator factor data. clinic index can be designed flexibly, clinic index includes physician, date, diagnosis.

The data update frequency control table is further set with a cold data interval. The data retrieving module first determines whether the data in the required data located in the hot data interval and the cold data interval have all been stored as the target medical structured data in the information database 110, if not, then obtain the data that has not been stored and store it as the target medical information database 110. The structured data in the target medical information database 110, if it is, then only obtain the data in the hot data interval in the required data, and Store it as structured data in the target medical information database 110, or update the plurality of structured data stored in the target medical information database 110 accordingly.

The data retrieving module 120 according to a hot data interval of the medical quality indicator configured in the data update frequency control table, determines whether to obtain only the data located in the hot data interval in the required data, or obtain the required data according to the update frequency The data in data is located in the hot data interval.

Data sources include: information room database (mainly associated data tables), reporting system (abnormal events, indicators of various departments, etc.), clinical indicator event collection system. The data retrieving module 120 will be accorded to each medical indicator database Sources, each design a data reading interface (such as SQL syntax, API interface, etc.) to interface with retrieving data. After collecting the data, the data retrieving module 120 can accord to the characteristic requirements of the indicator and set the data conversion method. Data is converted from different sources and different formats into the same format, and then used in subsequent models. In other words, modules in subsequent stages, regardless of the source of data collection, will accept data that has been converted and in the same format. The data retrieving module 120 can adjust and determine the data retrieval and the update frequency of the data retrieving module 120 based on the unique life cycle and timeliness of each indicator through a modular approach, taking into account data immediacy, accuracy and maintaining reasonable data maintenance costs.

The data retrieving module 120 will convert the original scattered information data into a after data collection according to the operational definition of the indicator, through data type definition, data content calculation, field naming conversion and data type integration, etc. The data is completed, the consistency of the data is ensured, and then the data is output to the target medical information database performs for storage.

In terms of quality management indicators, the data retrieving module 120 can create a specific index: all indicator factors follow the “patient”, and the patient's data in the hospital is based on “visit” (outpatient clinic, emergency department, emergency room, etc.) hospitalization) as the index. Therefore, establishing a data set of “patient-clinic index-indicator factor” is most conducive to subsequent data analysis and application.

When the data retrieving module 120 stores the information of “patient-clinic index-indicator factor”, the record creation time, change time, and operator will be appended at the same time to facilitate subsequent inspection and proofreading.

The data retrieving module 120 ensures the timeliness and accuracy of data in a staged temporary storage (staging) manner, and at the same time provides subsequent subgroup analysis (such as departmental analysis, age and gender stratified analysis, or downward, defines additional descriptive information other than indicator factor when analyzing to the patient level).

The data analyzing module 130 according to the operational definition performs data confirmation and data exclusion on the plurality of structured data, and according to the viewing request performs a computation on the plurality of structured data, to obtain a set of statistical data. The data analysis module 130 mainly performs the two major tasks of data confirmation and computation. As to data collection, different information databases may have different naming and recording methods for the same data due to their respective establishment needs, or has similar recording methods for different data. Therefore, the data analyzing module 130 will perform merging, confirmation and exclusion of the collected data based on the operational definition of medical quality control indicators, and only retain the required data and ensure after correctness, the computation is performed. When integrating computation, it is necessary to clearly define how the data is divided on the timeline. Traditional indicators are mostly in the form of monthly reports, that is, most indicators are calculated once every month. However, the data analysis module 130 can perform planning based on the needs of different time and space backgrounds and indicator operational definitions, at least based on day, week, month, quarter, year and other different cycles of integrated computation methods, giving full play to the scalable medical quality indicator management. The advantages of the system and its operating methods achieve the goal of a smart indicator system. The presentation method of monthly reports mainly meets the needs of paper printing layout, and the medical department performs multiple customized groups, which at least includes the entire hospital, departments of internal medicine, departments of surgery, gynecology and pediatrics, emergency and family medicine, facial features and other groups, each group includes multiple clinical units of medical departments at all levels.

The data visualization module 140 displays the set of statistical data through a user interface. The visualization platform has various modes, common ones such as Microsoft Power BI, SAS Visual Analytics, Tableau, Qlik View, etc. Different visualizations Platforms have different interface requirements. The data visualization module 140 is mainly used as a communication interface for data to interact with users. It can be designed according to the user's purpose requirements and the characteristics of the visualization platform to interface the data side with the visualization platform to present data to achieve features that can be expanded to different usage needs and platform system requirements. In the future, if the indicators have been informed to a certain extent, smart applications can be made in the visualization module, such as: outlier warnings, etc., and if the content of the indicators can Then the user will view and collect feedback information, so that further corrections and improvements can be made to the system itself in the future.

The data visualization module 140 includes a plurality of interface means, which at least includes API communication interface. The visualization platform includes a plurality of presentation methods, which at least includes monthly report and dashboard mode. The presentation mode of the dashboard is in line with the computer screen operation Mainly, and provides multiple interactive query interfaces, which at least includes items such as this month's summary, trends in recent years, dynamics in the last 30 days, monthly query, quarterly query, pivot analysis, indicator description, etc. Each item on the dashboard includes the drill-down function can query the detailed data of multiple “patient-clinic index-indicator factor”. Because the original data has been recoded and stored in the target medical database, each user has different needs (for example: by year/quarter/monthly overview or drill-down to each case) can be used as an index through indicator factor to quickly obtain each of the related patient-clinic index-indicator factor data, the data and represents each drill-down case in different Indicator results generated at time points. At the same time, the time information is also included in the data, so it can be quickly sorted and classified according to year/quarter/month performances.

In this way, the operation of the KPI indicator through the indicator basic data-cold hot data interval-upstream data-execution record is used as a new index code for the management indicator in the automated operation of the information system. Each indicator has its own unique corresponding unit. Operation index, through the index can quickly understand and manage the operation of the current indicators. Through the definition and regulation of the cold hot data interval, unnecessary data retrieval can be effectively reduced. Taking the patient examination critical value response rate as an example, if the original Each time the annual data is queried, nearly one million pieces of data need to be mobilized. Regardless of the cold hot data range, whether each query is annual or N-year data, only the data of the past 7 days will be mobilized from the original database, which averages about 2,000, and can effectively improve system performance and ensure system stability.

The mission scheduling module 150 arranges the data retrieving module 120 to retrieve or update data for multiple medical quality indicator performs. The mission scheduling module 150 has a mission description list and a schedule control unit. The mission description list records the multiple medical quality indicators each have the update frequency, retry frequency, hot data interval and upstream job name. The schedule control unit according to the mission description list triggers the data retrieving module 120 performs data retrieval or update. The content of the job is on the main server (such as medical operating system), query and reorganize the data, and then upload the data to the designated visual data platform. The name of such work item is “the name of the indicator report for performing data search and sorting”. In the data update frequency control table the update frequency and hot data intervals for all indicators can be viewed and managed.

The frequency of the automatic update data of the work is preset to automatically update every 8 hours, through the automatic update frequency, real-time data can be obtained.

After the indicator work fails to be executed, the default is to wait for 1 hour and then try again.

The number of days that the work needs to continuously update data (hot data) is preset to 7 days. This hot data interval helps to quickly generate report data.

If the reference base date is set to 0, it means that the base date is today. If it is set to −1, it means that the base date is yesterday.

If the data collection start date is set to 2020 Jan. 1, it means that data collection will begin on Jan. 1, 2020.

The upstream job name refers to the indicator name (job name) of the upstream data, and is defaulted to a null value.

In each loop execution, the mission scheduling module 150 checks whether there is a job that meets the execution conditions. If so, the job is executed. The condition judgment process includes:
1. Load basic settings: Read the above mission description list, load the work setting content and create a work status file to record the work execution. At the same time, performs basic data check, includes: data type definition (numeric type, Bollinger type, date and time type), check the name of the upstream indicator (if there is an upstream indicator specified, check whether the upstream database exists; if not, perform the upstream database creation work first), check necessary parameters (check the frequency of indicator updates, Try the basic settings such as frequency and time unit again. If not filled in, the work will report an exception and record it in the work status file).
2. Calculate the data demand interval: The calculation work should be the time when the execution starts and the date interval of the data that needs to be retrieved. According to the hot data period, the data collection start date and the date base offset number of days, obtain should be the existence target medical data date in information database.
3. Create a to-do list: Compare the data date in the target medical information database with the previously calculated data date, and create a to-do list (really performs to retrieve the data date list).
4. Performs data capture and record execution status: According to the above to-do list, performs data capture work. At the same time, record the time point of data capture, calculate the time point when the next loop starts execution, and whether the capture work is successfully completed. Record the information on the work status table.
5. Loop: If the system does not restart, after completing the performs data retrieval and recording the execution status, the system will wait until the time reaches the expected execution time of the next job, and then performs data retrieval and recording. Log the status. If the system restarts, start again from the step of loading the basic configuration.

For example, there is now work item 1. When the system starts executing, it will first check whether the indicators the update frequency and retry frequency and time unit are filled in. If there is no problem, perform step a.

The system according to the date base offset is 0, the data collection start date is Jan. 1, 2019 (2019-01-01), compare the data dates in the target medical information database, and determine which data are If it does not exist, then create a to-do list and performs data retrieval work. If there are missing settings, the system will alarm and stop execution; if the settings are correct, then create a to-do list.

According to the to-do list performs data retrieval, if the to-do list is empty, it means that all data already exists. Then the hot data of step a under performs is updated. The hot data interval is set to 2, and the system only will update data (data within 2 days) on today and yesterday. Determine whether the system execution is successful. If successful, update hot data (data within 2 days) a time every 8 hours. If the system execution fails, it needs to wait 2 hours and try again to update hot data (data within 2 days). The next start time is calculated, and then the steps are repeated to determine whether the system execution is successful.

For example, there is work item 3 now. If the indicator is executed successfully, it will be updated a time every 24 hours. If the execution fails, it will be executed again after two hours. The hot data interval is 2, and only the data of the last two days will be updated at this time. The date base offset is 0, and the starting date of data collection is 2022 Jan. 1. This means that the data in the database will have all the data starting from 2022 Jan. 1 till today. The upstream data is work item 1, so when performing data retrieval of work item 3, priority will be given to confirmation of data of work item 1.

For example, there is work item 2 now. When the system starts executing, it will first check whether the update frequency and retry frequency and time unit indicators are filled in. If there is no problem, perform step a. There is work item 1 to be performed and checked whether data already exists.

The system according to the date base offset is 0, and the data collection start date is 2022 Jan. 1. The data date in the target medical information database is compared to determine which data does not exist, and then to-do list is created and data retrieval is performed. If there are missing settings, the system will alarm and stop execution. If the settings are correct, a to-do list will be created. According to the above to-do list the data retrieval is performed. If the to-do list is empty, which means that all data already exists. Then the hot data update in step a is performed. The hot data interval is set to 2, and the system each time will only update data (data within 2 days) on today and yesterday. The system is determined whether the execution is successful, if successful, hot data will be updated once every 24 hours (data within 2 days). If the system fails to execute, it needs to wait 2 hours and try to update hot data (data within 2 days) again. The next start time is calculated, and then the steps are repeated to determine whether the system execution is successful.

Without using hot data and upstream data, work item 1 captures data once every 8 hours, each time starting from 2019. Work item 2 captures data once every 24 hours. Each time the data of work item 1 starting in 2022 must be obtained first, and then the data of work item 2 starting in 2022 must be obtained. Work item 3 must obtain data once every 8 hours, and each time it needs to first obtain the data of work item 1 starting in 2023, and then to retrieve the data of work item 3 starting in 2023. Every time the data is updated, all data from the start date to the present date needs to be retrieved.

In the case of using hot data and upstream data, work item 1 only needs to be retrieved from 2019 for the first time (when the target medical information database does not have data). After that, each update only needs to performs data retrieval to the missing data (to-do list). If there is no missing data, only hot data (2 days) performs retrieval is required each time. Both work item 2 and work item 3 are directly imported into the upstream data (work item 1 exists in target medical information), there is no need to re-create all the data of working item 1. For working item 2 and working item 3, only the first time (when the target medical information database has no data) needs to start from the start day of the configured data (work item 2 is 2022 Jan. 1, work item 3 is 2023 Jan. 1) for retrieval, and each subsequent update only needs to perform data retrieval for the missing data (to-do list); if there is no missing data, only hot data (2 days) needs to be retrieved each time.

In order to manage the operation of many KPI indicators, the present invention provides a work management system and method, which can be used to process complex work flows and perform different types of work. The invention makes work management more convenient, controllable and Efficient, while improving the performance and reliability of applications. This invention has broad application prospects and is of great value in the fields of automation, data processing, workflow management, etc. It can extract and merge data from different sources into patients, clinic index and indicator factor. This data structure integrates different related information into a, and stores it in the target medical information database for subsequent use.

Through the index method composed of basic indicator data, cold hot data interval, upstream data and execution records, the status and settings of all KPI indicators are managed. Through the index, the relevant information of different KPI indicator work is integrated together. Including job name, status, settings, etc., thereby providing a unified index to manage jobs. At the same time, according to the settings and status performs calculation of each KPI indicator job, the priority of the job, dependencies and other factors are taken into account to determine the best execution time and ensure that the work can be executed as expected. Through the execution record, the status of the work is updated and a Boolean value is returned, which represents whether the work was successfully executed and the updated work status.

### Example 1: Scalable Medical Quality Indicator Data Collection and Management System in Hospitals

A scalable medical quality indicator data collection and management system in hospitals, includes a data retrieving module, which includes a scalable database retrieval interface, and a reading interface for different medical indicator database interfaces to retrieve data, the reading interface at least includes SQL syntax and API communication interface. At the same time, it at least defines data type, data content calculation, field naming comparison and data type integration, etc., and converts it into a complete data; a data storage module, It converts the complete data after the data retrieving module to create a data set of “patient-clinic index-indicator factor”. At the same time, it meets the needs of subsequent analysis modules to provide extensible definitions and provides additional indicators other than factor for each data definition. The information described is output and written to the destination database. The data analysis module, which performs data confirmation and exclusion post-performs analysis, at least includes department analysis, age and gender analysis, stratified analysis, patient level analysis, etc. At the same time, performs integrated computation Separated from the data timeline, this module can perform integrated computation according to the needs of different time and space backgrounds and indicator operational definitions. The data visualization module receives the data transmitted by the data push module, and presents the data through the visualization platform.

Taking the inpatient mortality rate as an example, its operational definition is as follows.

The data collection scope of the indicator: includes (1) acute general hospital beds, (2) special intensive care beds, burn beds, infant beds, cribs and subacute respiratory care beds (RCC), burn intensive care beds, bone marrow transplants Hospital beds and isolation beds.

The indicator factor: The denominator of this indicator is the number of patients discharged from all the aforementioned beds (including transfers) in the monitoring month. Each patient should be classified into one of the following status performs: (1) Death discharge, (2) Critical illness automatic discharge: the doctor determines that the patient is critically ill and is on the verge of death, and the patient's family requests automatic discharge, (3) automatic discharge against medical advice (AAD), (4) direct discharge (MBD)

Exclusion rules: You can use the “filter by index case admission conditions” function to switch cases that exclude cases in the baby room (department code: NB), psychiatric ward (department code: PSY, GPSY, PSYD), and hospice ward (department code: HOSP).

### Calculation Formula:

In-hospital mortality rate (including voluntary discharges in critical condition)=Number of deaths (including voluntary discharges in critical condition)/total number of discharges

In-hospital mortality rate (excluding voluntary discharges due to critical illness)=number of deaths (excluding voluntary discharges due to critical illness)/total number of discharges.

Following on from above, the data retrieving module includes 4 control items: data access method control table, data update frequency control table, data computation logic control table, data output format comparison table. the data retrieving module through data access method control table, can interface with different data sources, includes relational database, such as Oracle, SQL Server, DB2, etc.) and supports operations such as ETL, extract, transform and load through SQL syntax that performs data; or is a file type data source, such as: Excel file, TXT file, XML file and other data sources. The data retrieving module can connect multiple data sources through multiple methods described in item 2, and multiple terminal settings. With Taking the operational definition of inpatient mortality rate as an example, the data retrieving module will retrieve includes (1) patient basic data file, (2) patient hospitalization login database, (3) physician data file, (4) patient hospitalization history database, etc. At least 4 data sources to meet the requirements of operational definition.

The data source can be defined as cold hot data according to the usage requirements, through the data update frequency control table defines the data update frequency and cold hot data interval. Through the following pseudocode method is used to determine whether the data is new data every time the data is updated. If it is new data, regardless of cold data or hot data, it will be output to the destination database for storage through the data computation logic control table and data output format comparison table. If it is old data, it will be further determined whether it is cold data or hot data. If the old data and are cold data, data processing will not be performed to improve the performance of data processing. If the old data and are hot data, then the data will be output to the destination database after processing through the data computation logic control table and the data output format comparison table. Taking the inpatient mortality rate as an example, the inpatient death data that exceeds a month does not have much change in actual business, so the interval of hot data is defined as the data within 30 days from the date of data capture. Data older than 30 days is defined as cold data.

An example of virtual code (pseudocode) is as follows:

```
cold data interval = 2020-01-01 ~ the previous 30 days
hot data interval = previous 30 days ~ today
Required data date list = [day for day in date_range(2020-01-0, today)]
Existing data date list = Read and listdatabase existing file date
for data date in required data date list:
  if data date not in existing data date:
    data type = new data
    data needs to be processed and stored
  elif data date in existing data date:
    data type = old data
    if data date incold data interval:
      data not processing
    elifdata date hot data interval:
      data needs to be processed and stored
else:
      raiseException( )
```

The data output format comparison table divides the data format definition into three types, namely numeric, datetime, and string. Through the following pseudocode method, all input data formats are mapped to three types data type.

An example of virtual code (pseudocode) is as follows:

```
List of numerical data types = [‘n’, ‘number’, ‘float64’......]
List of date data types = [‘d’, ‘dt’, ‘datetime’, ‘datetime64[ns]’......]
List of string data types = [‘s’, ‘str’, ‘string’, ‘object’......]
if input data format in date class data type list:
  Output data format = pd.to_datetime (input data) →Date data format output
elif input data format in numerical data type list:
  Output data format = pd.to_numeric (input data) • Numeric data format output
elif input data format in string class data type list:
  Output data format = input data.astype(‘object’).fillna(‘’) →String data format output
else:
  raiseException( )
```

The data storage module records the data type provided by the data output format comparison table, names and integrates multiple custom fields, performs data, and outputs data in a standardized format and stores it in the destination database. Data output format comparison the format of the tables is “data field name=data type code”, and the data set structure of “patient-clinic index-indicator factor” is created.

Taking the mortality rate of inpatients as an example: it is necessary to obtain the dates of each hospitalization and discharge of the patient, the department of each hospitalization, the bed number and the status of each discharge (dead, alive or dying, etc.), in order to According to operational definition performs calculation and exclusion. Therefore, the data set structure and examples of its “patient-clinic index-indicator factor” are as follows:

Patient data: should include at least medical record number, hospitalization number, name, gender, and birthday.

Clinic index: should include at least outpatient, inpatient, and emergency.

Indicator factor: should include at least hospitalization date, length of stay, discharge date, discharge time, discharge status, attending physician code, attending physician name, department code, ward number, bed number.

Additional information: includes data creation time, data change time, operator.

TABLE 1
data set architecture

| Data | | Data type | Example(Complete |
|---|---|---|---|
| architecture | Data column | code | data record) |
| Patient | Clinic number | String(s) | 10****40 |
| | Admission number | String(s) | 28****96 |
| | Name | String(s) | *** |
| | Gene | String(s) | M |
| | Birth | Date(d) | 19**/**/** |
| Clinic index | Clinic index | String(s) | Admission |
| indicator factor | Admission date | Date(d) | 2022 Aug. 29 |
| | Admission time | Date(d) | 16:04:00 |
| | DischargeDate | Date(d) | 2022 Aug. 30 |
| | Discharge time | Date(d) | 11:05:00 |
| | Discharge main | String(s) | Discharge |
| | status | | |
| | Discharge | String(s) | Back home |
| | secondary status | | |
| | Discharge detail | String(s) | Back home then go |
| | status | | clinic |
| | Main doctor | String(s) | DOC2***** |
| | number | | |
| | Main doctor name | String(s) | *** |
| | Department code | String(s) | GNS |
| | Room number | String(s) | A1** |
| | Bed number | Value(n) | 1** |
| | indicator factor | String(s) | Mortality |
| Additional | Data establish time | Date(d) | 2022 Aug. 30 |
| information | Data modify time | Date(d) | — |
| | Operator | String(s) | QAC8*** |

The data analyzing module can record the plurality of complete data, performs confirmation and exclusion functions of data according to plural conditions, which at least includes the condition confirmation and missing value exclusion of indicators. and can performs a plurality of integrated computations, it at least includes the integration of computation function by dividing the performances by daily, weekly, monthly, quarterly, annual and other periods. Taking the inpatient mortality rate as an example, the data analysis module can perform the following indicators for the indicators:
(1) Integrated computation based on performs in time periods (year, quarter, month, week, day), for example: display the mortality rate in a certain year, the mortality rate in a certain month, and the inpatient mortality rate in the past two years (2020˜2022) wait.
(2) Integrated computation of performs by department, for example: display the mortality rate of inpatients in the Department of Internal Medicine or the mortality rate of inpatients in the Department of Orthopedics and Cardiology.
(3) Integrated computation based on attending physician performances, for example: mortality rate of inpatients treated and cared for by a certain physician.
(4) Integrate calculations of the above different conditions, for example: the inpatient mortality rate of orthopedics in 2022 (integrated by time and department), the inpatient mortality rate of doctor A's treatment and care in the past two years, and by month performs (multiple integrations with doctors performs in time)

The following table is an example of integrating computation based on the ear, nose, throat, head and neck of a certain year and a certain month (multiple integrations based on time and department performances).

TABLE 2
Integrated computation results

| | | | | | | Death number | Death rate | |
|---|---|---|---|---|---|---|---|---|
| | | | | Death | Death | (including | (excluding | |
| | | | Discharge | number(including | rate(excluding | critical | critical | Discharge |
| Year | Month | Department | department | critical AAD) | critical AAD)* | AAD) | AAD)* | number |
| 20 | ** | ENT head | Rhinology, Head | *** | *.**% | *** | *.**% | *** |
| ** | | and neck | and Neck Surgery | | | | | |
| 20 | ** | ENT head | Laryngology and | *** | *.**% | *** | *.**% | *** |
| ** | | and neck | Neck Surgery | | | | | |
| 20 | ** | ENT head | otology | *** | *.**% | *** | *.**% | *** |
| ** | | and neck | | | | | | |

The data visualization module includes a plurality of interface means, which at least includes an API communication interface. The visualization platform includes a plurality of presentation methods, which at least includes a monthly report and a dashboard mode. The presentation method of the monthly report is consistent with the paper column The printing layout needs are mainly based on the needs of the printing layout, and the medical department performs multiple customized groups, which include at least the whole hospital, internal medicine department, surgical department, gynecology and pediatrics, emergency and family medicine, facial features and other groups, each group includes A plurality of medical department clinical units at all levels. Example: The internal medicine department group includes units such as internal medicine department, thoracic department, etc., and the internal medicine department includes a level two units such as general internal medicine, nephrology department, etc. The dashboard is presented in a manner that matches the computer screen Mainly operations, and provides multiple interactive query interfaces, which at least include items such as this month's summary, trends in recent years, dynamics within the last 30 days, monthly query, quarterly query, pivot analysis, indicator description, etc. Each item on the dashboard is includes the drill-down function, which can query the detailed data of multiple “patient-clinic index-indicator factor”. Taking the inpatient mortality rate as an example, the drill-down function provides detailed data of each data field in the data set table. You can Allow users to review detailed records of each data transaction.

### Example 2: Specific KPI-Patient Examination Critical Value Response Rate

In order to ensure “hospital medical quality and patient safety”, our hospital stipulates that doctors should use official mobile phones, hospital websites (SMSOT), or medical within 24 hours after receiving abnormal patient test results or critical notification text messages. Operating system report query reply.

When the laboratory completes the test report, if the test result is abnormal, the piece of data will be recorded in the Abnormal Report Registration (ARR); and then through a to-many approach, corresponding to the patient's For multiple doctors such as attending physicians, residents and billing physicians, the data will be recorded in the Order Abnormal Report Notification (ARN). Then a text message will be sent through the text message sending system to notify all doctors recorded in the ARN. On the mobile phone, data such as the time and content of the text message sent are recorded in the text message paging log file (PHSLOG). After the doctor receives the text message, the content and time of the reply text message through the official mobile phone will be recorded in the reply text message content file (PHSREPLY). If the doctor responds through the hospital website (SMSOT) or medical operating system performs, it will be recorded in the outlier notification record table (ORDREPLY) data file.

Following on from the above, to monitor the response rate of patient test critical values, it is necessary to obtain data from at least 5 databases: Abnormal Report Registration (ARR), Abnormal Report Notification (ARN), SMS Paging log file (PHSLOG), text message reply content file (PHSREPLY) and outlier response record table (ORDREPLY). If you want to further understand the doctor's data and patient's data, you need to add at least two more databases: doctor's basic data file and patient Basic data file.

That is, our hospital has an average of more than 10,000 abnormal reports every month. Each report corresponds to at least 3 doctors, and each doctor has three possible reply methods, which amounts to 90,000 reports. If the amount of data is further expanded to quarterly or annual data, the amount of data retrieved each time will exceed several million. If further classified according to the department to which the physician belongs and the patient's basic data performs, at least 2 more databases must be connected in series, and the amount of data will also increase multiple times. If such a huge amount of data is frequently retrieved from the information system, it will cause a high burden on the system operation. Therefore, most of them are current month's data is calculated once a month to maintain the stability of the system. However, many patients with abnormal tests may have irreparable consequences due to lack of proper processing in a short period of time. Therefore, in order to ensure medical quality and patient safety, the indicator should be a real-time reflection of the current situation, rather than a lagging indicator that only performs statistically once a month.

From the original database according to the definition of indicator factor, retrieve the data and convert it into the content of indicator factor required, establish the patient-clinic index-indicator factor, that is, retrieve the data from the original medical information database through the patient medical record number (such as The above 7 databases obtain data), and mainly use the patient's medical record number with clinical index (physician, date, test order number) and other record data, and according to the inspection item order number is connected to the indicator factor (test abnormal value response rate) and store this file in the destination medical database.

The original data is scattered in 7 original medical information databases. Each database cannot be connected from beginning to end. It needs to be connected through different indexes. This also means that every query needs to search 7 from beginning to end. All contents in a database, which results in limited system performance and inability to perform frequent queries in real time. REQNO will be generated after the inspection is issued, and the index can correspond to the inspection outlier registry (ARR). The inspection outlier registry (ARR) Then through ARRID corresponds to the test abnormal value notification table (ARN), and then through ARNID corresponds to the abnormal job response record table (ORDREPLY). The patient's basic data file and the physician's basic data file correspond to the test order through HISID as an index. Test order Then through REQNO is concatenated with the SMS notification record, and then through MSGID is used as an index to correspond to the SMS reply content file.

According to the present invention, the data performs of the aforementioned seven databases are sorted and concatenated, then recoded and stored in the target medical database. The data will be as follows, through patient-clinic index-indicator factor as the target medical database. New index encoding for data:
[Patient] Medical record number (patient's basic data file)-Test order number (patient's basic data file)-
[Clinic index] Inspection date (patient's basic data file)-physician (physician's basic data file)-
[Indicator factor] Inspection report results (abnormal value registration form)—report date (abnormal value registration form)—abnormal order number (abnormal value registration form)—abnormal content (abnormal value notification form)—SMS notification time (SMS paging record file))—SMS content (SMS paging record file)—Physician reply method (SMS reply content file/abnormal value reply record table)—Reply content (SMS reply content file/abnormal value reply record table)—Reply time (SMS reply content file/Outlier response record table)—reply time difference.

At the same time, because the original data has been recoded and stored in the target medical database, even if the needs of each user will be different (for example: overview by year/quarter/month or drilling down to each case). You can use indicator factor as an index to quickly obtain each of the relevant patient-clinic index-indicator factor data, the data and represent the indicator results generated by each drill-down case at different time points. At the same time, in the data It also includes time information so it can quickly perform sorting according to year/quarter/month.

There are many KPI indicators required in hospital management, and each indicator has its own particularity, which determines the update frequency of each indicator and its correlation with each indicator. Use the patient check critical value to reply For example, the hospital can stipulate that after receiving a text message notifying a patient of abnormal test results or critical status, the hospital should use a business mobile phone, the hospital website (SMSOT), or the medical operating system to report a query and reply within 24 hours. That is to say, as long as After the SMS notification is sent more than 24 hours, regardless of whether the doctor responds, the record will be regarded as no reply. At the same time, the hospital will open a 7-day monitoring period. Although there is no reply within 24 hours, there will be a reply within 7 days. record.

From the above definition we can set the operating characteristics of the indicator:
1. Cold data is the abnormal record of all checks recorded by the system so far (it has been more than 7 days, so it does not need to be updated every time).
2. Hot data is the data record within 7 days (it is necessary to confirm whether there is a reply).
3. The update frequency of hot data (once every 8 hours).

The present invention uses the operation of the KPI indicator through the indicator basic data-cold hot data interval-upstream data-execution record as a new index code for the automated operation of the management indicator in the information system:
[Indicator basic data] Indicator name—whether the indicator is activated—current activation status
[cold hot data interval] cold data interval-hot data interval—the update frequency—
[Upstream data] Whether there is an upstream indicator—the name of the upstream indicator
[Execution record] Last start execution time—Last end execution time—Last execution result—Next start execution time—Other notes

Each indicator has its unique corresponding single-a operation index, through which the index can quickly understand and manage the operation of the current indicator. Through the definition and regulation of the cold hot data interval, unnecessary data retrieval can be effectively reduced to Take the patient examination critical value response rate as an example. If it is necessary to mobilize nearly a million pieces of data each time the annual data is queried, through the cold hot data interval, no matter whether the annual or N-year data is queried each time, only the original data will be queried. The database mobilizes the data of the past 7 days, with an average of more than 2,000 data, which can effectively improve system performance and ensure system stability.

In order to manage the operation of many KPI indicators, the present invention provides a work management system and method, which can be used to process complex work flows and perform different types of work, making work management more convenient, controllable and efficient, while improving Application performance and reliability. The invention has broad application prospects and is of great value in the fields of automation, data processing, workflow management, etc., such as:
(1) Extract and merge data from different sources into a patient-clinic index-indicator factor data structure. This data structure integrates different related information together, and stores it in the target medical information database for subsequent use.
(2) Through indicator basic data-cold hot data interval-upstream data-execution record index method to manage the status and settings of all KPI indicators. through the index integrates the relevant information of different KPI indicator work, including Job name, status, settings, etc., thereby providing a unified index to manage jobs. At the same time, according to the settings and status performs calculation of each KPI indicator job, taking into account the priority of the job, dependencies and other factors, to Determine the best execution time and ensure that the work can be executed as expected. Through the execution record, update the status of the work and return a Boolean value, representing whether the work was successfully executed and the updated work status.

The medical quality indicator management system and method provided by the present invention can efficiently share information in different reports, thereby saving a large amount of computation resources, and appropriately optimize computation resources to increase the frequency of automatic update of reports, so a This can significantly reduce the cost of managing reports.

While preferred embodiments of the present invention have been shown and described herein, it will be obvious to those skilled in the art that such embodiments are provided by way of example only and can be implemented in combinations. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for managing a medical quality indicator, comprising:
a target database for medical data;
a data retrieving module connected to the target database, wherein
in response to a request for viewing a medical quality indicator or based on an update frequency of the medical quality indicator set in a data update frequency control table, the data retrieving module obtains required data from one or more primary database for medical data according to an operational definition of the medical quality indicator, and preprocesses the required data and stores it as a plurality pieces of structured data of the medical quality indicator in the target database, each piece of the structured data correspondingly storing a patient data of a patient, a clinical index data of the patient and an indicator factor data of the medical quality indicator; and
the data retrieving module, based on a hot data interval of the medical quality indicator set in the data update frequency control table, determines whether only a piece of data of the required data present in the hot data interval is obtained, or obtains a piece of data of the required data present in the hot data interval according to the update frequency;
a data analyzing module connected to the target database, which performs a data confirmation and data exclusion with respect to the plurality pieces of structured data according to the operational definition, and performs a computation to the plurality pieces of structured data according to the viewing request to obtain a set of statistical data; and
a data visualization module connected to the data analyzing module and used for displaying the set of statistical data through a user interface.

2. The system of claim 1, wherein the operational definition defines one or more data items required for evaluating the medical quality indicator, and the data retrieving module obtains the required data from one or more corresponding primary databases according to the data items.

3. The system of claim 1, wherein the patient data includes medical record number, hospitalization number, inspection sheet number, name, gender, birthday, or a combination thereof.

4. The system of claim 1, wherein the clinical index data includes outpatient, emergency department, inpatient, or a combination thereof.

5. The system of claim 1, wherein a cold data interval is further set in the data update frequency control table, and the data retrieving module first determines whether both a piece of data of the required data present in the hot data interval and a piece of data of the required data present in the cold data interval are stored as structured data in the target database, if negative, then obtains data not stored stores it as structured data in the target database, and if affirmative, then obtains only the piece of data of the required data present in the hot data interval, and stores it as structured data in the target database, or accordingly updates the plurality of structured data stored in the target database.

6. The system of claim 1, further comprising a mission scheduling module connected to the data retrieving module and used for arranging the data retrieving module's data retrieval or update with respect to a plurality of medical quality indicators, the mission scheduling module having a mission description list and a schedule control unit.

7. The system of claim 6, wherein the mission description list records an update frequency, a retry frequency, a hot data interval and an upstream mission name for each of the plurality of medical quality indicators.

8. The system of claim 6, wherein the schedule control unit activates the data retrieving module to perform data retrieval or update according to the mission description list.

9. A method for managing a medical quality indicator, comprising:
providing a target medical information database, a the data retrieving module, a the data analyzing module and a the data visualization module, wherein the data retrieving module is connected to the target medical information database, the data analyzing module is connected to the target medical information database, the data visualization module is connected to the data analyzing module;
using the data retrieving module to obtain required data from one or more original medical information database corresponding to a viewing request of a medical quality indicator, or according to a the update frequency of the medical quality indicator configured in a data update frequency control table, according to an operational definition of the medical quality indicator, after a data processing, storing the required data as the plurality of structured data of the medical quality indicator in the target medical information database, wherein each of the structured data correspondingly stores a patient data of a patient, a clinic index data of the patient and an indicator factor data of the medical quality indicator;
using the data retrieving module to determine whether only a data located in the hot data interval in the in the required data is obtained according to a hot data interval of the medical quality indicator configured in the data update frequency control table, or a data located in the hot data interval in the required data is obtained according to the update frequency;
using the data analyzing module to perform data confirmation and data exclusion to the plurality of structured data according to the operational definition, and perform a computation to the plurality of structured data according to the viewing request to obtain a set of statistical data; and
using the data visualization module to display the set of statistical data through a user interface.

10. The method of claim 9, wherein the operational definition defines one or more data items required for evaluating the medical quality indicator, and the data retrieving module obtains the required data from one or more corresponding primary databases according to the data items.

11. The method of claim 9, wherein the patient data includes medical record number, hospitalization number, inspection sheet number, name, gender, birthday, or a combination thereof.

12. The method of claim 9, wherein the clinical index data includes outpatient, emergency department, inpatient, or a combination thereof.

13. The method of claim 9, wherein a cold data interval is further set in the data update frequency control table, and the data retrieving module first determines whether both a piece of data of the required data present in the hot data interval and a piece of data of the required data present in the cold data interval are stored as structured data in the target database, if negative, then obtains data not stored stores it as structured data in the target database, and if affirmative, then obtains only the piece of data of the required data present in the hot data interval, and stores it as structured data in the target database, or accordingly updates the plurality of structured data stored in the target database.

14. The method of claim 9, further comprising a mission scheduling module connected to the data retrieving module and used for arranging the data retrieving module's data retrieval or update with respect to a plurality of medical quality indicators, the mission scheduling module having a mission description list and a schedule control unit.

15. The method of claim 14, wherein the mission description list records an update frequency, a retry frequency, a hot data interval and an upstream mission name for each of the plurality of medical quality indicators.

16. The method of claim 14, wherein the schedule control unit activates the data retrieving module to perform data retrieval or update according to the mission description list.
